(19) United States
(12) Patent Application Publication
(10) Pub. No.: US 2024/0339204 A1
(43) Pub. Date: Oct. 10, 2024

(54) MODULAR MEDICAL STATION WITH ACCESS CONTROL AND EXPANDABLE CAPABILITIES

(71) Applicant: GoForward, Inc., San Francisco, CA (US)

(72) Inventors: Adrian Aoun, San Francisco, CA (US); Taylor Roan, San Francisco, CA (US)

(21) Appl. No.: 18/586,423

(22) Filed: Feb. 24, 2024

Related U.S. Application Data
(60) Provisional application No. 63/494,962, filed on Apr. 7, 2023.

(51) Int. Cl. G16H 40/20; G16H 10/60; G16H 15/00; G16H 40/40; G16H 40/67; G16H 50/20

(52) U.S. Cl. CPC G16H 40/20; G16H 10/60; G16H 15/00; G16H 40/40; G16H 40/67; G16H 50/20

(57) ABSTRACT

Embodiments relate to a medical station deployable at various locations with expandable plug-in software and hardware components. The medical station includes access control features that limit a person's entry for use as well as restricting software or hardware components that the person may use. Actuators in the medical station are operated so that the person may use the components in a timely and adequate manner. Some of the actuators are provided in drawer assemblies that are operated to provide the person or human assistant access to medical supplies or tools.

### CROSS-REFERENCE TO RELATED APPLICATIONS

This application claims priority under 35 U.S.C. § 119 (e) to U.S. Patent Application No. 63/494,962, filed on Apr. 7, 2023, which is incorporated by reference herein in its entirety.

### BACKGROUND

Embodiments relate to modular medical stations and a system including the same for expedited and efficient deployment at various sites.

In order for a patient to receive medical diagnosis, treatment or consultation, the patient generally visits a medical facility such as a hospital. Such medical facility may be located away from the patient's home or workplace. A visit to the medical facility may sometimes involve an extended amount of time to travel to the facility. Hence, it would be desirable to have medical facilities deployed across various sites. However, setting up the medical facilities at different sites involves a large amount of investment due to the use of large space and construction costs as well as employment of medical professionals at each of the sites.

To reduce costs and facilitate medical attention to many patients, virtual sessions with medical professionals via video conference or emails are becoming more popular. However, such virtual sessions are often restricted to unavailability or limited availability of medical equipment at the patient's location. Hence, only limited types of medical services may be provided to the patient via the virtual sessions.

### SUMMARY

Embodiments relate to a medical station that selectively enables a person's entry into its interior and launches one or more applications selected by the person to receive medical services. Input associated with the person is received whether the person has authorization to access the medical station. The medical station selectively enables the person to enter it when the person is determined to have authorization to access the medical station. After entering the medical station, the person may select the application to be launched on the medical station. After launching the selected application, the application may grant the person access to use a medical tool or a component in the medical station.

In one or more embodiments, a control signal is received after launching the application. When the control signal is received, one or more actuators in the medical station are operated to grant the person access to the medical tool or the component.

In one or more embodiments, a drawer or a tray is activated by the one or more actuators when the control signal is received.

In one or more embodiments, a visual indicator associated with the activated drawer or the tray is turned on when the control signal is received.

In one or more embodiments, a plurality of applications are stored in the medical station. A subset of applications accessible by the person according to the person's authorization to access is determined, and the subset of applications is presented to the person for selection.

In one or more embodiments, another medical tool or another component is stored in the medical station. The access to the other medical tool or the other component is restricted to operation of another application.

In one or more embodiments, the applications are associated with different medical providers.

In one or more embodiments, the applications are received from a computing device located remotely from the medical station via a communication network.

In one or more embodiments, whether the received applications satisfy predetermined requirements associated with the medical station is determined. One or more of the received applications that satisfy the predetermined requirements are installed in the medical station.

In one or more embodiments, the predetermined requirements indicate installation of a corresponding medical tool or a corresponding component in the medical station.

In one or more embodiments, the person is enabled to enter the medical station by opening or unlocking a door to enter the medical station.

In one or more embodiments, measurements on the person are taken by using sensors in the medical station.

In one or more embodiments, a sequence of questions is presented to the person by the application. A sequence of actions is taken by the application. The sequence of actions includes the use of the medical tool or the component in the medical station.

In one or more embodiments, information associated on the person or treatment being performed on the person is collected using the medical tool or the component. The collected information is sent to a computing device located remotely from the medical station via a communication network for analysis and processing.

In one or more embodiments, a medical record of the person in the computing device is updated according to the collected information.

Embodiments also relate to a medical station that has a restricted entrance that selectively enable a person to enter its interior. The medical station includes an interface device for receiving input associated with authorization to access the medical station. A computing device in the medical station receives the input from the interface device, and enables the person to enter the restricted entrance by granting access to the interior of the medical station when the person is determined to have authorization to access the medical station. The computing device also receives selection from the person of the first application to be launched on the medical station, and grants access to the person to use a medical tool or a component in the medical station after the launching of the selected application.

### BRIEF DESCRIPTION OF THE DRAWINGS

Figure (FIG. 1 is a block diagram illustrating the overall network environment including medical stations, according to one embodiment.

FIG. 2 is a block diagram of a medical station, according to one embodiment.

FIG. 3 is a block diagram illustrating software modules in memory, according to one embodiment.

FIG. 4 is a block diagram illustrating components of a backend server, according to one embodiment.

FIG. 5 is a perspective view of the medical station, according to one embodiment.

FIGS. 6A and 6B are diagrams illustrating pop-up drawers with medical tools, according to one embodiment.

FIGS. 7A and 7B illustrate example user interface screens of the medical station, according to one embodiment.

FIGS. 8A and 8B illustrate example external structures of the medical station, according to one embodiment.

FIG. 9 illustrates an example interior structure of the medical station, according to one embodiment.

FIG. 10 is a flowchart illustrating operations at the medical station, according to one embodiment.

### DETAILED DESCRIPTION OF EMBODIMENTS

Embodiments are described herein with reference to the accompanying drawings. Principles disclosed herein may, however, be embodied in many different forms and should not be construed as being limited to the embodiments set forth herein. In the description, details of well-known features and techniques may be omitted to avoid unnecessarily obscuring the features of the embodiments. In the drawings, like reference numerals in the drawings denote like elements. The shape, size and regions, and the like, of the drawing may be exaggerated for clarity.

Embodiments relate to a medical station deployable at various locations with expandable plug-in software and hardware components. The medical station includes access control features that limit a person's entry for use as well as restricting software or hardware components that the person may use. Actuators in the medical station are operated so that the person may use the components in a timely and adequate manner. Some of the actuators are provided in drawer assemblies that are operated to provide the person or a human assistant access to medical supplies or tools.

FIG. 1 is a block diagram illustrating the overall network environment 100 of medical stations 140, according to one embodiment. Network environment includes a backend server 120, service developer devices 130 and medical stations 140 that communicate over the network 110. Although service developer devices 130 are illustrated in FIG. 1 as communicating directly with backend server 120, service developer devices 130 may communicate with backend server 120 and/or medical stations 140 via network 110.

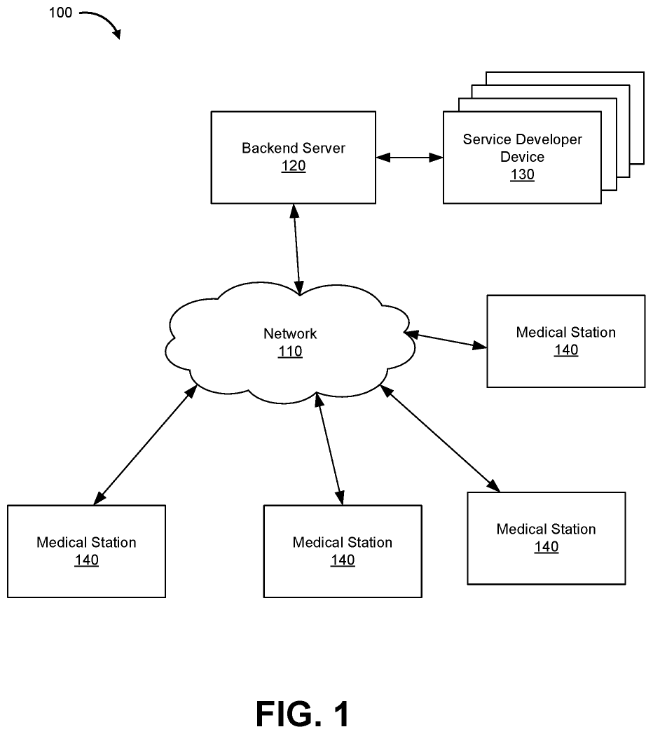

A medical station described herein is a prefabricated modular structure equipped with sensors and medical tools to provide medical services to patients. Medical station 140 may have a predetermined dimension, such as 10 feet by 15 feet or any other suitable dimensions, that can be installed inside or outside a building. An example structure of medical station 140 is described below in detail with reference to FIG. 2. Medical station 140 communicates with backend server 120 to send and receive login information of the patient accessing medical station 140, send and receive information associated with applications launched at medical station 140, and send information on inventories of medical tools in medical station 140.

Due to a relatively small size and the ease of installation, medical stations 140 may be beneficially installed at diverse geographical locations at relatively low cost. Medical stations 140 may be mass produced at a factory and have forms and structures adapted for deployment with reduced construction fees. A patient may visit one of many sites installed with medical stations to receive medical services.

In one or more embodiments, medical stations 140 may be operable shortly after plugging them into power outlets and setting up wireless or wired communication. Hence, compared to weeks or months of time for construction and configuring of equipment at medical facilities, medical stations 140 may be deployed and operated within a day or even hours. Furthermore, medical stations 140 can be relocated to another site with ease, depending on, for example, changing needs at different sites. It should be noted that in some embodiments, the medical stations 140 may have independent power sources, such as solar panels, generators, fuel cells, and the like. Likewise, the medical stations 140 may have self-contained wireless communications systems built-in, obviating the need for separately coupling to communication interfaces prior to deployment and operation of the medical stations. In some embodiments, the medical stations 140 may be deployed outdoors, may be mobile or otherwise movable (e.g., via built-in wheels or other transportation infrastructure), may be self-leveling (e.g., via one or more position or orientation adjustment mechanisms), and/or may be pre-assembled or pre-manufactured for on-site assembly.

More than one medical station 140 may be installed at the same site to expand the capacity or service capabilities at the location. For example, two or more medical stations 140 with the same configuration may be installed at the same site (e.g., a shopping mall) so that multiple patients can simultaneously receive medical services. Alternatively, medical stations of different configurations may be installed at the same site so that the patients may receive different types of medical services at the same site. For example, a medical station may be customized to diagnose or treat cardiovascular disease whereas another medical station may be customized to diagnose or treat mental issues. For each customization, each medical station may have different medical tools and/or plug-in components available for use by the patients. Such plug-in components may be hardware components or software components.

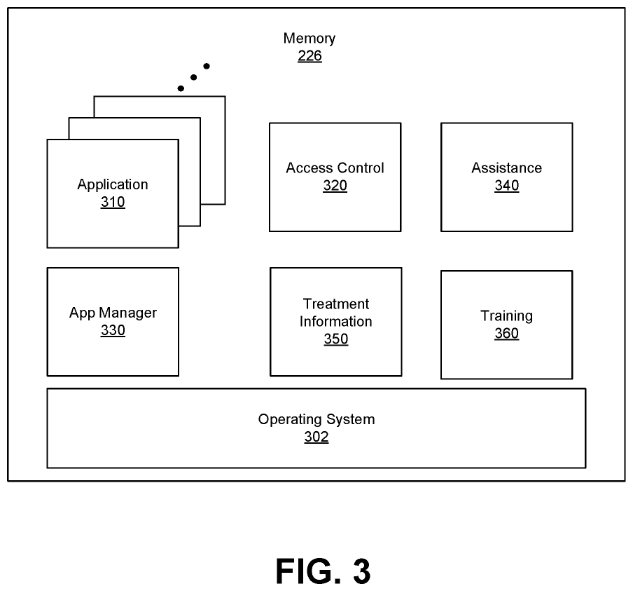

Backend server 120 is a computing device that facilitates or supports medical station 140 to provide medical services to patients. Backend server 120 may store various applications from service developer devices 130 and send the applications to medical stations 140 for deployment. Backend server 120 may also evaluate performance of various services provided via medical stations 140 by collecting information received from medical stations 140, and also take actions to manage medical stations 140. The components and functions of backend server 120 are described below in detail with reference to FIG. 4.

Service developer devices 130 are computing devices for developing and/or researching medical services provided using medical stations 140. For this purpose, service developer devices 130 may receive information on current and past patients' health conditions, treatments received by the patients, and other sources and execute specialized algorithms. By receiving and processing such information, service developer devices 130 may update parameters of applications executable on medical stations, and develop or assess treatment protocols.

Service developer devices 130 may be operated by the same entity that operates medical stations 140. Alternatively, each service developer device 130 may be managed and operated by an entity different from the entity that operates medical stations 140. In such cases, the entity managing or operating a service developer device may pay the entity operating medical stations 140 fees for deploying its application and enabling patients to use its services on medical stations 140. Alternatively, these entities may enter into a fee sharing arrangement to share fees collected from the patients or their health insurance companies. Different entities may also purchase, license or lease the medical stations and pay fees to access and use service developer devices 130 or develop their own platform and implement service developer devices 130 to operate in a manner compatible with their business models. Various types of arrangements may be made between manufacturers of the medical stations, service developers, insurance companies and other payors and patients accessing the medical station 140.

Network 110 is a set of devices that enable computing devices to communicate with each other. Various communication protocols may be used by network 110 including, but not limited to, Internet Protocols, Ethernet and wireless network protocols such as cellular standards.

FIG. 2 is a block diagram of medical station 140, according to one embodiment. Medical station 140 may include, among other components, computing device 220, one or more display devices 230, input devices 232 for receiving user input, drawer assemblies 240, sensors 250, network interfaces 260 and plug-in components 270. Medical station 140 may include other components not illustrated in FIG. 2 such as a turntable on which a patient stands for scanning of the patient's body with a body scanner, interfaces that enable third-party or modular equipment or sensors to couple with and provide data/power to and from the equipment, and any other suitable equipment that extends the functionality of the medical station 140.

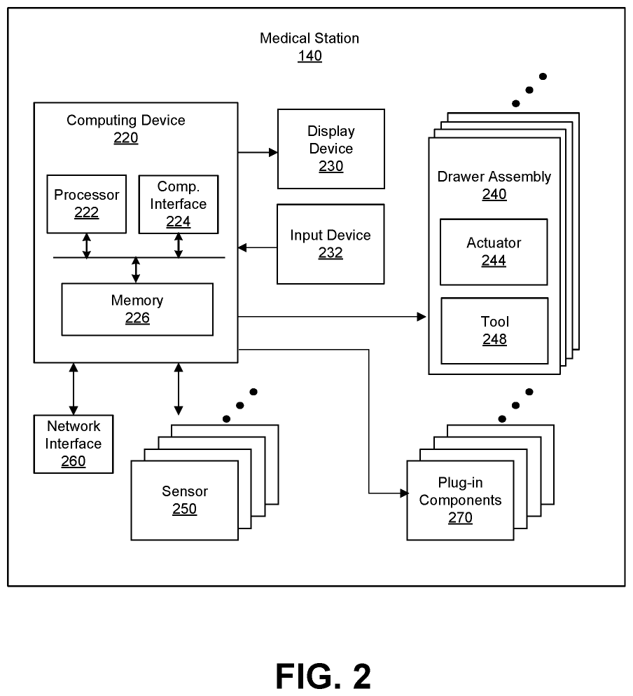

Computing device 220 executes logic to the interface and controls other components of medical station 140. For this purpose, computing device 220 may include, among other components, processor 222, component interface 224, and memory 226. Processor 222 is a hardware circuit or hardware circuit in combination with firmware that executes instructions stored in memory to perform various functions on computing device 220. Memory 226 is a non-transitory storage medium for storing software modules executable on the processor 222. The details of the software modules in memory 226 are described below in detail with reference to FIG. 3. Component interface 224 is hardware or hardware in combination with software that enables interfacing with medical tools 248 (as applicable), sensors 250, and/or plug-in components 270. Component interface 224 may include multiple subunits that enable a computing device 220 to communicate with different components of the medical station 140 over different protocols.

Display device 230 receives display signals from a computing device 220 to display various images to a patient or a human assistant. In one embodiment, one of the display devices 230 may be a touch screen that is embedded onto a wall of medical station 140. Multiple display devices may also be included in a medical station 140. For example, a display device may be installed at the entry of a medical station 140 to display information related to access to the medical station 140 and/or the types of medical services provided by the medical station 140 while another device installed in the interior of the medical station 140 may display graphical user interfaces to select and receive a specific medical service.

Input device 232 is a device for receiving user inputs. Input device 232 may be embodied as part of a display device (e.g., a touch sensor) or a separate component (e.g., a microphone, a mouse or a keyboard). More than one input device may be included in medical station 140 to support different types of user inputs.

Drawer assemblies 240 are mechanically operated drawers that enable a patient or a human assistant to selectively access a medical tool or medical supplies. A drawer assembly may be locked or opened through the operation of actuator 244 that operates according to a control signal from a computing device 220. Some of the drawer assemblies may have different sizes (e.g., height and width) to facilitate the user to identify the right drawer assembly for use and accommodate tools of different dimensions. In addition or alternatively, some or all drawer assemblies may include a visual indicator (e.g., lighting elements) that is turned on when selected for use by an application executed on a computing device 220. By turning on the visual indicator, the user may conveniently identify the drawer assembly that includes medical tools or medical supplies that are relevant to actions to be taken by the user. Computing device 220 may also keep track of drawer assemblies that were previously opened so that the same medical tool used may not be reused on another patient for hygiene reasons.

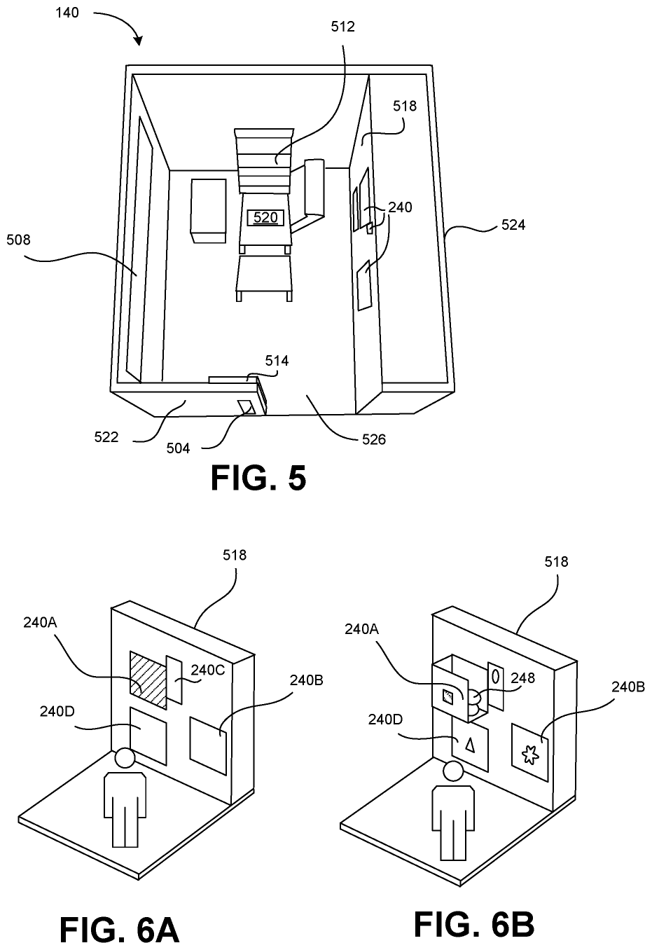

In one or more embodiments, the inventories of medical tools in a medical station 140 may be tracked and managed by backend server 120, as described below in detail with reference to FIG. 4. An example of operating drawer assemblies and their configuration is described below in detail with reference to FIGS. 6A and 6B. Medical tools 248 may include, among others, a wireless heart rate monitor, digital stethoscope, contactless thermometer, pap smear kit, blood draw kit, vaccine supplies, ear lavage kit, dermatoscope, or any other suitable device or equipment for self-administration or administration by a healthcare provider. At least some of these medical tools 248 communicate via a component interface 224 to enable collection of patient information in real-time while other medical tools 248 are collected for further testing and analysis. In some embodiments, the drawer assemblies 240 function as an API, allowing third-party entities to interface with the medical station 140 by, for example, specifying drawer requirements (such as a size, temperature control, power requirements, and the like) for providing medicine, treatments, testing equipment or other medical or wellness equipment to an individual.

Sensors 250 are provided in the medical station 140 to detect physical characteristics or positions of the patients. In one embodiment, sensors 250 may be embodied as a body scanner that detects dimensions of various parts of the patient's body. The body scanner may be used in conjunction with a turntable (not shown) that rotates the patient while the scanning is being performed and measures the patient's weight. A glucose meter may also be provided in a medical station 140 as one of the sensors 250 to detect the patient's blood sugar level with or without drawing blood from the patient. A chair in a medical station 140 may include one or more sensors for detecting the presence or the pose of the patient sitting on the chair. Additional sensors 250 can include a thermography sensor, a blood pressure measurement system, a blood oxygenation detection system, a heart rate monitor, a body position sensor, a glucose meter, or any other suitable sensor or measurement/detection system.

Network interface 260 is hardware or hardware in combination with firmware that enables a medical station 140 to communicate with a backend server 120 or service development devices 130 via a network 110. Network interface 260 may, for example, include an antenna and circuits for communicating over the Internet via wireless communication and/or a network card for communicating via wired communication (e.g., Ethernet) or for communication via private networks or peer-to-peer.

Plug-in components 270 are physical components added to a medical station 140 to expand functionality to the medical station 140. Plug-in components 270 may include a digital stethoscope, a digital dermatoscope, a pap smear kit, an electrocardiogram machine (e.g., EKG machine), an ultrasound device, a spirometer, a retinoscope, a blood draw kit, treatment kits (e.g., a cryo-gun, an ear lavage kit, a surgery kit, and the like), exercise materials (such as resistance bands), food and drinks, merchandise, prescription medicine, over-the-counter medicine, educational or informative materials, and the like. Functionalities of the medical station 140 that may leverage plug-in components 270 include but are not limited to: generating a 3D body model, measuring blood oxygenation, measuring/listening to heart sounds, performing venipuncture or other blood collection, measuring weight or height, administering vaccines or other injections, performing a nasal swab, collecting a urine sample or other specimen, measuring blood pressure, observing the insides of a patient's ears/nose/mouth/throat, capturing images of a patient's skin, performing an ultrasound/x-ray/MRI on a patient, observing a patient's eyes (e.g., using an ophthalmoscope), performing an EKG, performing a CT scan, performing spirometry functions, analyzing a patient's posture, performing cardiac telemetry, performing tonometry functions, capturing retinal or other images of the patient's eyes or other organs/body parts, capturing thermal or hyperspectral images of the patient, and analyzing a patient for chemicals or other compounds.

Plug-in components 270 communicate with computing device 220 using any combination of wired & wireless signals, such as WiFi, Bluetooth, radio frequency, cellular networks, satellite, infrared, mmWave, USB, serial, ethernet, fiber optic, direct signal wires, etc. whether directly, or via the internet. This may also include manual methods of conveying information from the plug in components to the station, such as data entry via touchscreen, keyboard, controller, and/or voice as input by the patient and/or the provider. Furthermore, plug-in components 270 may have dedicated power connections to provide power to the components from the station in some combination of wireless, conductor contact, and/or contact insertion interfaces for power transmission. These may be used to actively power a plug-in component, or even charge a component for use when repositioned.

FIG. 3 is a block diagram illustrating software modules in the memory 226 of a medical station 140, according to one embodiment. Memory 226 may store various software modules including, but not limited to, an operating system 302, applications 310, an application manager module 330, an access control module 320, an assistance module 340, a treatment information module 350 and a training module 360. Memory 226 may store software modules other than what are illustrated in FIG. 3, and one or more of the modules in FIG. 3 may be combined into a single module or split into further modules.

Applications 310 are software modules for interacting with the patients to provide medical or health related services to the patients. Each application 310 may be focused on different aspects of medical/health services (e.g., a mental health checkup service, and a cardiovascular health checkup service). In some embodiment, a different suite of applications developed and managed by different health care entities may be stored in memory 226. For example, different suites or sets of applications may be available to different patients depending on the health insurance plans of the patients, age/gender of the patients, membership levels of the patients or other criteria.

Some of the applications 310 may make one or more tools 248 in drawer assemblies 240 available to patients during their operations. For this purpose, the applications 310 may cause component interface 224 of computing device 220 to timely send activation signals to appropriate drawer assemblies 240 while the applications 310 are active. If multiple medical tools 248 are to be accessed by a patient, the active application may send a series of activation signals to activate the actuators 244 of relevant drawer assemblies 240 so that relevant medical tools 248 may be sequentially accessed by the patient. For example, when a patient activates a routine heath checkup application, the application may first provide a thermometer followed by a blood drawing tool and a urine sample bottle by sequentially operating drawer assemblies 240 with the relevant tools. Further, depending on the input from the patient or an input signal from a previous medical tool, the applications may vary the next medical tool to be subsequently accessed by the patient.

Applications 310 may also operate plug-in components 270 to perform further tests on the patient. The use of or access to certain plug-in components 270 may be restricted to a subset of applications. For example, a plug-in component specifically designed for a medical service provider may be accessed or used only by an application developed or affiliated with the same medical service provider. Such restriction to access and tallying of available tools may be implemented by access control module 320.

Application manager module 330 is a software module that manages deployment and availability of applications 310 on a medical station 140. For this purpose, application manager module 330 may communicate with backend server 120 to receive the most recent version of applications 310, deploy only applications that satisfy criteria applicable to the current medical station, and discard or delete applications that are out-of-date or not complying with regulations, agreements or other criteria. Such management operations may be performed periodically by application manager module 330, initiated through an update request received from backend server 120 or through maintenance personnel accessing the medical station.

In one or more embodiments, certain applications may be installed and deployed on a medical station only when certain requirements are satisfied. For example, an application may be installed only when the medical station has a certain plug-in component installed. Alternatively, certain applications may be installed only when the medical station is installed in approved geographical regions or sites or enabled only when a certified or skilled operator is present. Certain applications may also be installed and deployed for use in planning, scheduling and forecasting of visits by patients. Such restrictions may be enforced by application manager module 330. The restrictions as defined in application manager module 330 may be updated by backend server 120.

In some embodiments, the medical station 140 and/or the backend server 120 coordinate to implement control over which applications are installed at the medical station based on regulatory controls and requirements associated with the jurisdiction in which the medical station is located, based on staffing and training associated with the medical station, based on components and equipment available to the medical station, or based on any other suitable factor. In some embodiments, the medical station 140 itself doesn't host applications locally, but instead accesses applications stored within one or more cloud services. In some embodiments, applications that are installed within the medical station 140 will be limited by the capabilities offered by the applications depending on, for instance, the availability of equipment or personnel required to implement one or more functionalities of the applications.

Access control module 320 is a software module that controls access to the resources of a medical station. Access control module 320 may communicate with backend server 120 to enable only authorized patients to enter and use the medical station. Access control module 320 may also receive an electronic medical record (EMR) of the patient from backend server 120 or other sources, and provide information to applications 310 in a manner that complies with regulations and/or contracts. Access control module 320 may also restrict applications 310 from using certain medical tools 248 or plug-in components 270, depending on whether certain criteria are satisfied (e.g., patient having a certain membership level) and/or depending on whether an appropriately credentialed provider is present.

Assistance module 340 is a software module that coordinates with a human assistant (either onsite or remotely, live or asynchronously) to provide services to patients. Operations initiated by one or more applications 310 may involve assistance from a human assistant. Such operations may involve actions that are impractical or impossible to be legally performed at medical station 140 automatically without human assistance, and may include the manipulation of certain medical tools 248 or plug-in components 270. Assistance module 340 may also store a list of human assistants and their qualifications, and alert an appropriate human assistant among multiple human assistants to assist a patient. In one or more embodiments, applications 310 may, during their operations, instruct assistance module 340 to send out a request for a human assistant. In some embodiments, assistance module 340 may call or initialize robotic devices instead or in addition to requesting human assistance. Such robotic devices may, for example, perform operations such as drawing a blood sample from the patient or assisting the patient to take a certain position.

A treatment information module 350 is a software module for collecting patients' information associated with a treatment being performed on the patients. The patients' information may include treatments being received by the patients as well as various information of the patients before and after a treatment has been initiated. Such information may include medications being taken, physical therapies performed on the patients, dimensions/weights of patients, results from blood testing (e.g., blood sugar level), blood pressure measurements, response to treatments, etc. Treatment information module 350 may send the collected patients' information to a backend server 120 for further analysis and processing, as described below in detail with reference to FIG. 4. In some embodiments, a mobile device of a patient can interface with a medical station 140 or the backend server 120 to access patient information (such as treatment information, medical station use information, and the like) and present such information, for instance within an application running on the mobile device.

Training module 360 is a software module that instructs patients and/or human assistants to perform certain actions. The patients may be presented with video and/or audio files with instructions on how to operate medical tools 248 and/or plug-in components 270. The human assistants may also be presented with instructions to assist the patients during diagnostic or treatment actions performed on the patients within medical station 140. Some training instructions may be provided to human assistants while no patients are present in medical station 140. In some embodiments, the quality of human assistants or care providers is monitored to track the competencies of the individuals, which can be measured based on a comparison to other human assistants, based on a base-line set of evaluation criteria, based on user feedback, or based on any other suitable criteria. In some embodiments, the training module 360 can provide human assistants with information detailing user preferences (such as an arm preference, an aversion to needles, desired user position during action performed) or user history (such as past complications, past complaints, previous positive experiences, and the like). The training module 360 can leverage current best practices for particular care scenarios in real-time, beneficially enabling the medical stations to provide the best/preferred care at any given time. Further, data gathered from the medical stations 140 generally can enable the development and deployment of new care procedures, the standardization of care protocols, enabling consistent application of care, improving training protocols and procedures, and conforming standards of care to local care-oriented regulations.

Operating system 302 is a set of instructions for managing the resources of computing device 220. Example operating systems include Microsoft Windows, Unix, macOS, Linux, IOS, Android, etc. In addition, the operating system 302 can control medical station 140 lighting, sound (such as music, instructions, prompts, voice selection, etc.), logistics functions (such as medical station inventory, next patient steps, human assistant instructions, medical station or user monitoring, and the like), and automation functions (such as automated supply/capability management, automated lab processing, and the like).

FIG. 4 is a block diagram illustrating components of backend server 120, according to one embodiment. Backend server 120 may include, among other components, processor 402, memory 410, network interface 406 and bus 450 connecting these components. Backend server 120 may include other components not illustrated in FIG. 4.

Processor 402 reads and executes instructions stored in memory 226 to perform various operations on backend server 120. Although only a single processor 402 is illustrated in FIG. 4, multiple processors may be included in backend server 120.

Network interface 406 is hardware or hardware in combination with firmware or software for communicating with medical stations 140 and/or service developer devices 130. For this purpose, network interface 406 may implement various wired or wireless protocols.

Memory 410 is a non-transitory storage medium for storing software modules. Memory 410 may include, among other software modules, application repository 412, patient information module 420, medical station management module 430 and treatment assessment module 440. Memory 410 may store other software modules not illustrated in FIG. 4. Two or more software modules in FIG. 4 may be combined into a single module or a software module in FIG. 4 may be split up into multiple software modules.

Application repository 412 stores applications 416 to be sent to medical stations 140 for installation. Application repository 412 may retain the most recent versions of the applications 416 and may archive older versions of the applications 416. Newer applications or updated versions of the applications 416 may be automatically received from service developer devices 130 and be stored in application repository 412. In addition or alternatively, applications 416 may be transferred and stored in application repository 412 manually by human operators. Application repository 412 may also store metadata on applications 416, including but not limited to, related applications, stored versions of applications, ID of developers who developed the applications, and eligible membership to access the applications.

In other embodiments, backend server 120 does not include application repository 412. Rather, medical stations 140 receive applications directly from developers via network or through manual installation.

Patient information module 420 is a database including the list of patients and their membership information (e.g., name, age, gender, membership level, insurance information). Patient information module 420 may also include EMR of the patients that may be updated based on diagnostic operations and treatments performed at medical stations 140. Likewise, EMR or other patient information can be updated via a mobile device, via traditional medical facility visits, via a web interface, via a health app, or via any other suitable avenue. Patient information module 420 may be accessed by medical stations 140 to determine whether a patient should be granted access to medical stations 140 and/or certain applications 310 executable on medical stations 140.

Medical station management module 430 is a software module that performs various management operations related to medical stations 140. Medical station management module 430 may keep track of inventories of medical tools available in medical stations 140 and take actions to replenish them if the inventories are running low. For this purpose, medical station management module 430 may send orders to warehouses to ship tools 248 to sites where medical stations 140 are located. Also, medical station management module 430 may instruct maintenance personnel to visit and carry out predetermined maintenance operations on a certain medical station.

Treatment assessment module 440 performs analysis to determine efficacy of treatments performed on patients. For this purpose, treatment assessment module 440 may receive patients' information from one or more medical stations 140. Various statistical analyses may be performed on the patients' information to determine efficacy or performance of treatment options. In some embodiments, the distribution of multiple medical stations 140 as described herein allow users to access care more frequently than having to visit traditional doctor's offices, allowing for a greater collection of patient data. Likewise, the medical stations 140 described herein enable more and different types of patient data to be collected. The medical stations 140 can leverage this data to evaluate adherence to and effectiveness of particular treatment options, which in turn can be leveraged to evaluate best courses of action or treatment to recommend to other users. The patients' information may be provided in real-time to the treatment assessment module 440 to enable health service providers to evaluate, assess and choose treatment options. In addition, the patients' information (such as demographic or geographic information, health risks, existing conditions, history, and the like) may be leveraged to recommend certain health apps or programs offered by the medical stations 140.

The backend server 120 can access a number of datasets associated with a patient or with a medical station 140. For instance, the backend server 120 can access patient data from one or more medical data repositories, from applications run by the medical station 140 with which the patient interacts, from mobile applications (such as health or fitness applications) used by the patient via one or more mobile devices, from wearable devices worn or used by the patient, or from any other suitable source. The backend server 120 can likewise access data describing preferred treatment methods or standards of care, from medical or scientific literature, from government entities or agencies, from commercial data sources, or from any other suitable data source. The data accessed by the backend server 120 can then be leveraged to formulate treatment plans for a patient, to make recommendations to the patient, to inform one or more medical professionals about the patient, or for any other suitable use. In some embodiments, the accessed data (for instance, from a population of patients) can be used to train one or more machine-learned models, which in turn can be used to inform or predict treatment plans for future patients. Finally, the access data can enable the medical station 140 or the backend server 120 to confirm treatment plans prepared by medical professionals, and to flag anomalies within such treatment plans (such as treatment plans that don't conform to best practices as identified by the medical station or the backend server).

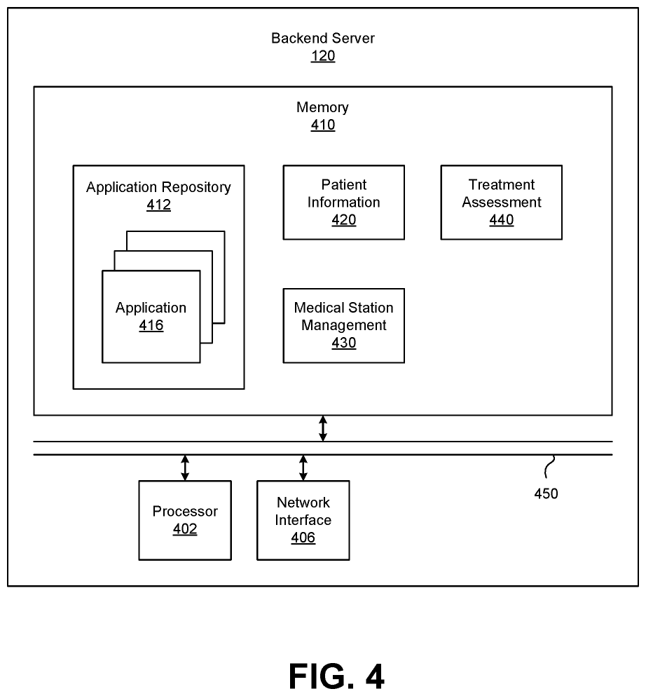

FIG. 5 is a perspective view of medical station 140, according to some embodiments. Medical station 140 may include a chassis 524 that forms an outer enclosure as illustrated in FIG. 5. The chassis 524 may be in the shape of a cube where one wall 522 has an entrance 526 with an automatic door 514, and another wall has a display screen 508. The display screen 508 may be a touch screen to allow the patient to provide answers or responses to displayed options.

A chair 512 is provided at one side of medical station 140. The patient may sit on the chair to perform various tests and provide feedback via input device 232. In one or more embodiments, plug-in components 270 may be attached to chair 512 to extend functionality of medical station 140. The chair 512 may include one more sensors 520 that detect the presence or the position of the patient on the chair 512.

Wall 522 is also provided with an interface 504 (e.g., a touch screen or console) at a side of the entrance 526 that enables the patient to input information and access medical station 140. The automatic door 514 attached to wall 522 may be opened only when computing device 220 determines that the patient has access rights or authorization to enter medical station 140.

Wall 518 is installed with drawer assemblies 240. Drawer assemblies 240 may be activated by the operation of an application 310 executed by medical station 140. FIGS. 6A and 6B are diagrams illustrating drawer assemblies 240 with medical tools 248, according to one embodiment. When application 310 determines that it is time to give patient 614 access to a specific medical tool, an actuator of a drawer assembly containing the specific medical tool operates to transition from a closed state (as illustrated in FIG. 6A) to an open state (as illustrated in FIG. 6B). To assist patient 614 to identify the correct drawer, a visual cue may be provided on the corresponding drawer assembly. The visual cue may include, for example, lighting up of an entire surface of a drawer (as illustrated in FIG. 6A by hatching pattern of drawer 240A) or a symbol on the surface (as illustrated FIG. 6B). It should be noted that although reference is made herein to drawer assemblies, in practice any delivery mechanism can be substituted in place of the drawer assemblies, including but not limited to cabinets, pockets, and the like. The selected drawer may be opened by manual operation after unlocking or it may be automatically opened by an actuator. After the drawer is opened, the patient, a human assistant or a robotic device may pick up the medical tools 248 in the drawer for performing subsequent operations on the patient.

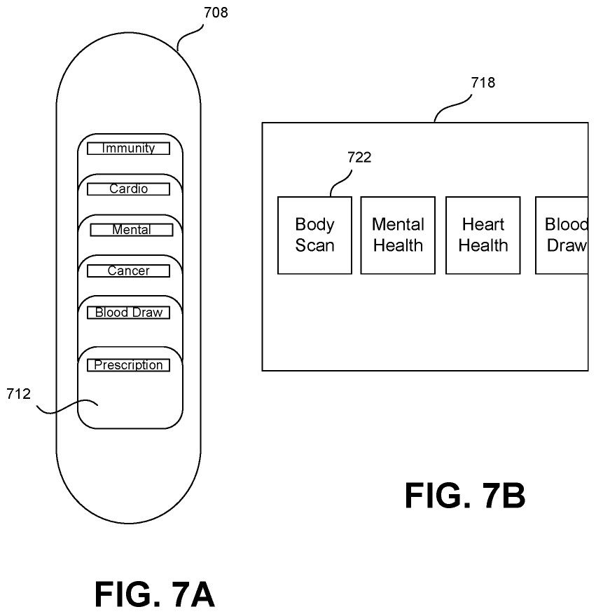

FIGS. 7A and 7B are diagrams illustrating the external structure of the medical station 140, according to one embodiment. FIGS. 7A through 9 illustrate example user interface screens, external structures, and internal structures of the medical station 140, according to various embodiments.

FIG. 7A is a user interface diagram illustrating a screen presented on a touch screen by the entrance door, according to one embodiment. FIG. 7B is a user interface diagram illustrating a screen presented on a display screen 508 on the wall inside the medical station, according to one embodiment. In FIGS. 7A and 7B, each box or icon represents an application that may be executed by the medical station 140 for use by a patient. The patient or human assistant may select one of the boxes or icons displayed on the screen to initiate an application relevant to the patient. The applicable boxes or icons to be presented on the display device may be selected and/or updated by access control module 320. The user input to select the box or icon may be received via touching of the screen, a voice command, a gesture or other modalities of user input.

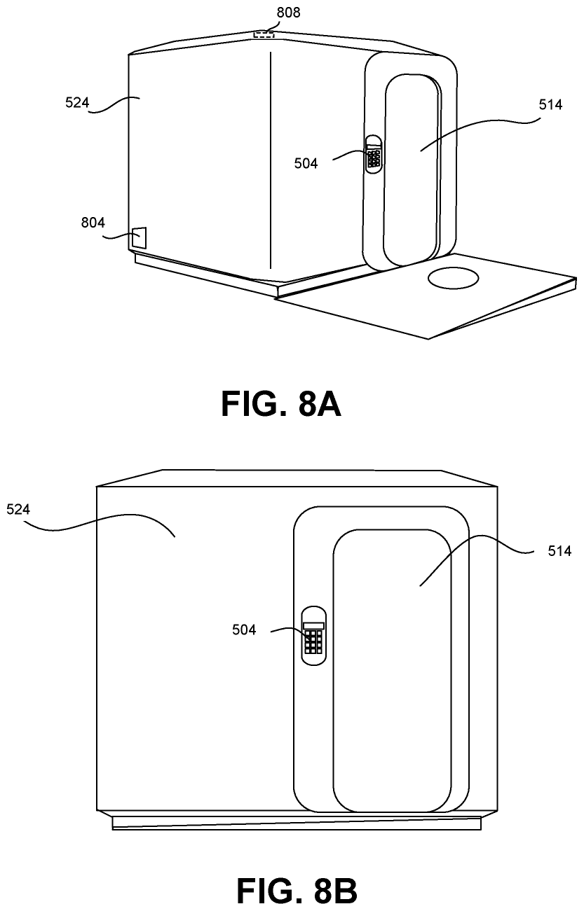

FIGS. 8A and 8B illustrate example external structures of the medical station, according to one embodiment. As shown in FIGS. 8A and 8B, the medical station 140 has an outer chassis 524, a door 514 and an adjacent door panel. The door 514 can be used by a patient to access the interior of the medical station 140, and the door panel can include an interface 504 that allows the patient to check-in to the medical station, to identify or authenticate themselves, to determine the capabilities of the medical station, to identify a functionality desired by or need of the patient, and the like.

The medical station 140 may include components for easy installation and deployment. Such components include a power plugin unit 804 and a communication system 808. The power plugin unit 804 may be connected to an outlet to receive power. In remote areas, the plugin unit 804 may be connected to a battery pack or a power generator. If access to a power grid is readily available, the plugin unit 804 may receive the power from the power grid. The communication system 808 is coupled to the network interface 406 to enable the medical station 140 to communicate with the backend server 120 or other external computing devices. The communication system 808 may be compatible with wireless or wired communication networks.

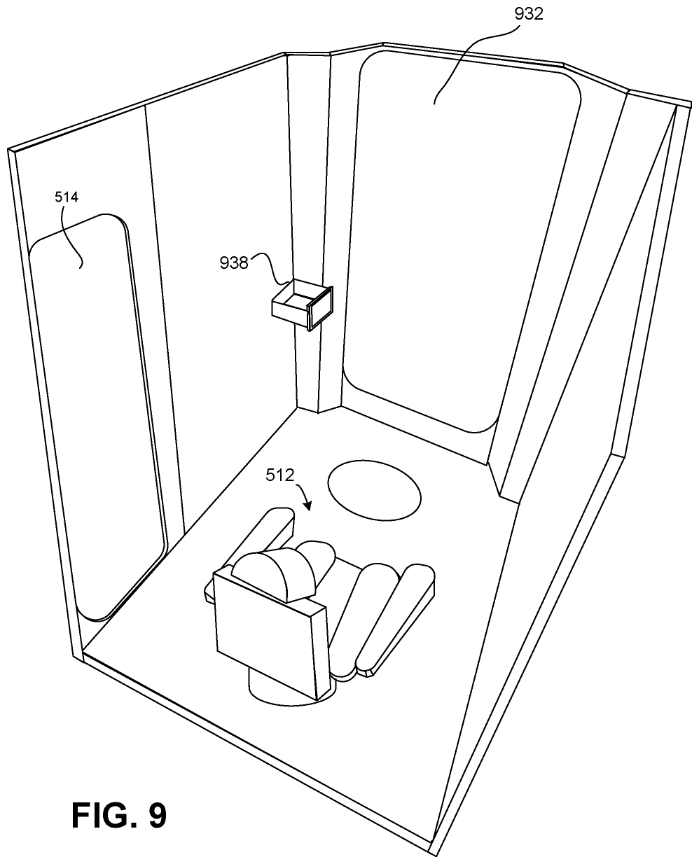

FIG. 9 illustrates an example interior structure of the medical station, according to one embodiment. As illustrated in FIG. 9, the medical station 140 interior includes a chair 512 that a patient can sit in, an interface 932 that a patient can interact with (e.g., via one or more applications executed by the medical station), and a tray 938 that can dispense one or more materials, inventories, medications, or other consumable or disposable items (for instance, a swab identified by the medical station as needed by the patient via one or more applications with which the patient interacts). The interface 932 may be embodied as a touch screen that displays images and also receives input from the patient. The tray 738 may be used in conjunction with or as an alternative to the drawer assembly described above with reference to FIGS. 5 through 6B. The components and their layout illustrated in FIG. 9 are merely illustrative and various other changes or additions may be made. For example, a body scanner may be provided in the medical station, and drawer assemblies may also be provided.

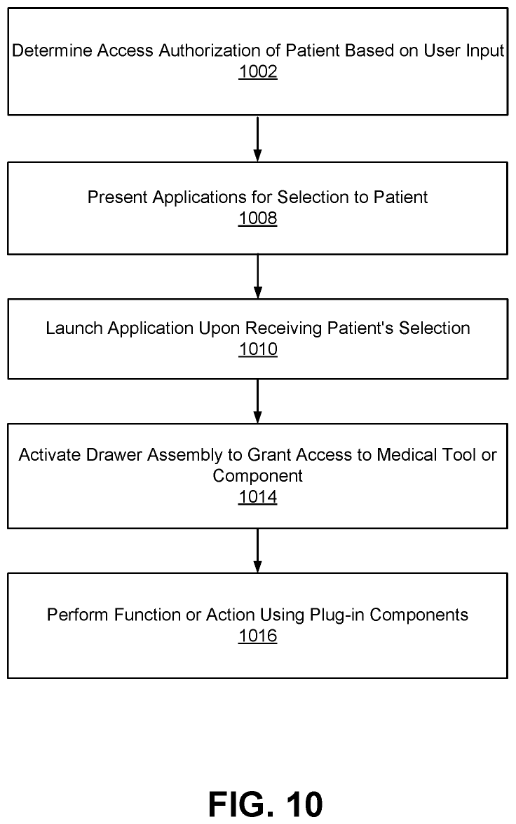

FIG. 10 is a flowchart illustrating operations of medical station 140, according to one embodiment. A patient may use the interface 504 of medical station 140 to provide user input for accessing medical station 140. The user input may include patient ID and password. The user input may be sent to backend server 120 via network 110 to determine 1002 the patient's access authorization or right by comparing the user input against entries in patient information module 420. In some embodiments, the patient may carry a radio frequency (RF) tag or other ID cards that may be read by the interface 504.

After the access to medical station 140 is confirmed at backend server 120, an entrance door to medical station 140 may be opened or unlocked to enable the patient to enter the interior of medical station 140.

Applications are then presented 1008 to the patient for selection. A plurality of applications selectable by the patient may be displayed on a display and the patient's selection may be received via a touch, a verbal command or a gesture. Instead of displaying the selectable applications, verbal prompts may be provided by a Text-to-Speech (TTS) system to enable the patient to make the selection of the applications.

Based on the selection of medical/health services, a corresponding application 310 may be launched 1010 by computing device 220 of medical station 140. After being launched, the application may take measurements on the patient using sensors 250 (e.g., a body scanner) in medical station 140. Some of these measurements may be taken by default whereas other measurements may be taken for only some patients with certain conditions. Other preliminary actions may be taken at medical station 140 along with taking the measurements, such as obtaining verbal approval on services to be provided by medical station 140 and receiving inputs from the patient on requested medical/health services. In some embodiments, such preliminary actions may be taken by a default application that is launched before or in conjunction with the launching of the application selected by the patient.

Further, application 310 may follow through a sequence of questions and take one or more actions to diagnose and/or treat the patient or collect and relay information to enable a healthcare professional to diagnose and/or treat the patient, which in some embodiments may or may not include questions asked by or actions performed by human medical personnel. As part of such series, one or more drawer assemblies 240 may be activated 1014 to enable the patient to access medical tools 248 included in the drawer assemblies 240. In order to facilitate the patient's use of the medical tools 248, instructions may be provided by activation of training module 360 on computing device 220.

Further, certain functions or actions may be performed 1016 in the medical station 140 by using 816 certain plug-in components. Assuming that the patient or selected services have access to the plug-in components, the launched application may cause computing device 220 to send signals to operate plug-in components.

The steps and their sequence as illustrated in FIG. 10 are merely illustrative. Additional steps may be taken by medical station 140 or certain steps may be omitted. For example, if medical station 140 is not equipped with any plug-in components, the process of using 1016 plug-in components may be omitted. Further, the sequence of activating 1014 the drawer assembly and using 1016 the plug-in components may be performed in parallel or in a reverse order.

In some embodiments, a medical station 140 may be identified or selected (for instance, via a mobile device application associated with the medical station) for use by the patient based on one or more of capabilities of the medical station, medical needs of the patient, characteristics (e.g., biologic, demographic, geographic, or any other characteristics of the patient), based on credentials of an operator or personnel associated with the medical station, based on availability/occupancy/use of the medical station, based on inventory available within the medical station, based on sensors or equipment available within the medical station, based on applications installed at the medical station, based on whether the patient has completed pre-requisite actions (e.g., filling out paperwork or disclosure forms in advance, fasting, taking medicines in advance, and the like), or based on any other suitable criteria. For instance, if a patient needs to have their blood pressure checked as part of a health screen, then a medical station 140 with blood pressure measurement capabilities can be selected and recommended for the user. In the event that a medical station 140 visited by a patient is unable to meet the needs of the patient, then an alternative medical station can be recommended to the patient based on the patient's needs.

The medical stations 140 described herein enable personalized care for a patient based on information associated with the patient that is available to the medical stations. By leveraging the scale of a network of medical stations and automating various processes and functionality, the cost of providing such care is generally reduced when compared to traditional access to healthcare (e.g., visits to doctor's offices). As noted above, the manufacture and assembly of such medical stations 140 can be streamlined, beneficially increasing the speed of deployment and lowering the barriers to creating a large, distributed network of deployed medical stations. Other functionalities of the medical stations 140 that may not already be described herein may include self-cleaning functionality, self-alerting functionality (to alert personnel or operators of the medical stations when inventory is low or particular functionalities of the medical stations aren't working properly), real-time inventory tracking and management (e.g., automated inventory ordering when certain supplies are low), modular installation (e.g., multiple medical stations can be installed side-by-side to increase throughput capacity), platform-oriented software deployment (e.g., an application can be built and targeted to all or a subset of a network of medical stations based on capabilities of the medical stations), and centralized regulatory compliance (e.g., the regulatory requirements of each jurisdiction can be centrally managed and enforced).

It should be noted that although the term “patient” is used herein, in practice, the medical station 140 can provide access to any number of health- or wellness-oriented services or functionalities (such as physical fitness, meditation, and the like), in which case a user of the medical station may not be a patient per se, but rather a consumer or participant in the capabilities of the medical station. Likewise, in practice, a version of the medical station 140 may not offer medical or wellness services or functionalities at all; accordingly, it should be noted that the features and functionalities described herein with regards to the medical station may apply equally to any station or system that provides users with access to one or more services or functionalities, such as a consumer activities, entertainment services, banking services, and the like.

Upon reading this disclosure, those of skill in the art will appreciate still additional alternative designs of the medical stations, their interoperating devices and operating schemes may be used. Thus, while particular embodiments and applications have been illustrated and described, it is to be understood that the invention is not limited to the precise construction and components disclosed herein and that various modifications, changes and variations which will be apparent to those skilled in the art may be made in the arrangement, operation and details of the method and apparatus disclosed herein without departing from the spirit and scope of the present disclosure.

1. A method of operating a medical station, comprising:
receiving input associated with a person to determine the person's authorization to access the medical station;
selectively enabling the person to enter an interior of the medical station responsive to determination that the person has authorization to access the medical station;
receiving selection from the person on a first application to be launched on the medical station; and
opening a drawer or a tray by operating one or more actuators in the medical station to grant the person access to a first medical tool or a first component responsive to launching of the selected application.

2. The method of claim 1, further comprising:
receiving a control signal responsive to the launching of the first application, wherein the one or more actuators are opened responsive to receiving the control signal.

3. (canceled)

4. The method of claim 2, further comprising turning on a visual indicator associated with the opened drawer or the tray responsive to receiving the control signal.

5. The method of claim 1, further comprising:
storing a plurality of applications in the medical station;
determining a subset of applications accessible by the person according to the person's authorization to access the subset of applications including the first application; and
presenting the subset of applications to the person for selection.

6. The method of claim 5, further comprising:
storing a second medical tool or a second component different from the first medical tool or the first component in the medical station; and
restricting access to the second medical tool or the second component to operation of a second application different from the first application.

7. The method of claim 6, wherein the first application and the second application are associated with different medical providers.

8. The method of claim 5, further comprising receiving the plurality of applications from a computing device located remotely from the medical station via a communication network.

9. The method of claim 8, further comprising:
determining whether the received plurality of applications satisfy predetermined requirements associated with the medical station; and
installing, in the medical station, one or more of the received plurality of the applications that satisfy the predetermined requirements.

10. The method of claim 9, wherein the predetermined requirements indicate availability of a corresponding medical tool or a corresponding component in the medical station.

11. The method of claim 1, wherein enabling the person to enter the medical station comprises opening or unlocking a door to enter the medical station.

12. The method of claim 1, further comprising taking measurements on the person using sensors in the medical station.

13. The method of claim 1, further comprising:
presenting a sequence of questions to the person by the first application; and
taking a sequence of actions by the first application including the use of the first medical tool or the first component in the medical station.

14. The method of claim 1, further comprising:
collecting information on the person or a treatment being performed on the person using the first medical tool or the first component; and
sending the collected information to a computing device located remotely from the medical station via a communication network for analysis and processing.

15. The method of claim 14, further comprising:
causing a medical record of the person in the computing device to be updated according to the collected information.

16. A medical station, comprising:
an interface device for receiving input associated with a person's authorization to access the medical station;
a restricted entrance configured to selectively enable the person to enter an interior of the medical station;
one or more actuators;
a drawer or a tray operable by the one or more actuators; and
a computing device configured to:
receive the input from the interface device,
enable the person to enter the restricted entrance by granting access to the interior of the medical station responsive to determining that the person has authorization to access the medical station,
receive selection from the person on a first application to be launched on the medical station; and
operate the one or more actuators to open a drawer or a tray and grant the person access to a first medical tool or a first component responsive to launching of the selected application.

17. The medical station of claim 16, wherein the computing device is configured to generate a control signal to operate the one or more actuators.

18. (canceled)

19. The medical station of claim 17, wherein the drawer or the tray comprises a visual indication that is turned on responsive to receiving the control signal from the computing device.

20. The medical station of claim 16, wherein the computing device is configured to:
store a plurality of applications in the medical station;
determine a subset of applications accessible by the person according to the person's authorization to access, the subset of applications including the first application; and
present the subset of applications to the person for selection.

21. The medical station of claim 16, further comprising a second medical tool or a second component different from the first medical tool or the first component in the medical station, wherein access to the second medical tool or the second component is restricted to operation of a second application different from the first application.

22. The medical station of claim 16, further comprising a communication system configured to receive a plurality of applications from a computing device located remotely from the medical station, the computing device further configured to install at least a subset of the received applications.

23. The medical station of claim 16, wherein the computing device is further configured to present a sequence of questions and take a sequence of actions by the first application including the use of the first medical tool or the first component in the medical station.

24. A non-transitory computer readable storage medium storing instructions thereon, the instructions when executed by a processor cause the processor to:
receive input associated with a person to determine the person's authorization to access a medical station;
selectively enable the person to enter an interior of the medical station responsive to determination that the person has authorization to access the medical station;
receive selection from the person on a first application to be launched on the medical station; and
operate one or more actuators to open a drawer or a tray and grant the person access to a first medical tool or a first component responsive to launching of the selected application.

25. The non-transitory computer readable storage medium of claim 24, further storing instructions that cause the processor to:
receive a control signal responsive to the launching of the first application, wherein the one or more actuators are opened responsive to receiving the control signal.

26. The non-transitory computer readable storage medium of claim 25, further storing instructions that cause the processor to turn on a visual indicator associated with the opened drawer or the tray responsive to receiving the control signal.
